(19) United States
(12) Patent Application Publication
(10) Pub. No.: US 2022/0288245 A1
(43) Pub. Date: Sep. 15, 2022

(54) RADIOPHARMACEUTICAL COMPOUND AND COMPOSITION FOR POSITRON EMISSION TOMOGRAPHY (PET) IMAGING OF INTERLEUKIN-2 RECEPTOR POSITIVE CELLS, PROCESS FOR THE PREPARATION THEREOF, RELATED KIT AND USES THEREOF

(71) Applicant: SAPIENZA UNIVERSITA' DI ROMA, Rome (IT)

(72) Inventors: Alberto SIGNORE, Rome (IT); Filippo GALLI, Rome (IT)

(73) Assignee: SAPIENZA UNIVERSITA' DI ROMA, Rome (IT)

(21) Appl. No.: 17/624,881

(22) PCT Filed: Jul. 2, 2020
(86) PCT No.: PCT/IT2020/050169

§ 371 Date: Jan. 5, 2022

(30) Foreign Application Priority Data
Jul. 5, 2019 (IT) 102019000011013

(51) Int. Cl. A61K 51/08; A61K 51/04

(52) U.S. Cl. CPC A61K 51/088; A61K 51/0478

(57) ABSTRACT

The present invention relates to a radiopharmaceutical compound or composition for Positron Emission Tomography (PET) imaging of interleukin-2 (IL2) receptor positive cells, in particular, ⁶⁸Ga-radiolabelled interleukin-2 such as a desalanyl-1, serine-125 human interleukin-2 (dsIL2) radiolabelled with a short-lived PET radioisotope (or radionuclide) gallium-68 (⁶⁸Ga) by using tris-(hydroxypyridinone-maleimide) (THP-mal) as a chelator. The invention concerns also a kit comprising dsIL2 linked to THP-mal which can be added with ⁶⁸Ga in order to obtain the above mentioned radiopharmaceutical, at room temperature, suitable for PET imaging, a process for the preparation of the radiopharmaceutical and its use in medical and diagnostic field.

The present invention relates to a radiopharmaceutical compound or composition for Positron Emission Tomography (PET) imaging of interleukin-2 (IL2) receptor (IL2R) positive cells, process for the preparation thereof, related kit and uses thereof. In particular, the present invention concerns a ⁶⁸Ga-radiolabelled IL2 such as a desalanyl-1, serine-125 human IL2 (dsIL2) radiolabelled with a short-lived PET radioisotope (or radionuclide) gallium-68 (⁶⁸Ga) by using tris-(hydroxypyridinone-malemide) (THP-mal) as a chelator. The invention concerns also a kit comprising dsIL2 linked to THP which can be added with ⁶⁸Ga in order to obtain the above mentioned radiopharmaceutical suitable for PET imaging, a process for the preparation of the radiopharmaceutical and its use in medical and diagnostic field.

Over the years many IL2 radiolabelling methods have been proposed, but none was suitable enough to translate its use in the clinics at low cost and with high efficiency.

Currently, the only way to detect activated IL2R positive cells in vivo is by performing a biopsy of the lesions of interest (when possible) and in vitro staining with specific monoclonal antibodies conjugated with a dye for immunohistochemistry or fluorescence. Of course, this procedure has many disadvantages since it is not always possible to perform biopsy and when possible, it provides only limited information.

In order to overcome the disadvantages of biopsy, attempts have been made using ¹²³I-labelled-IL2, ^{99m}Tc-labelled-IL2 or ¹⁸F-labelled-IL2, but the first two molecules did not allow PET use and the last one did not show good in vivo biodistribution together with a cumbersome labelling procedure.

For example, Technetium-99m labelled recombinant human IL2 (rhIL2) has been patented (U.S. Pat. No. 4,832,940), nevertheless, its chemical features, such as poor solubility in aqueous solvents and tendency to aggregate, makes it difficult and expensive to label it and use it clinically on a routine base. Despite numerous attempts, the realization of a formulation in the shape of a kit based on Technetium-99m-IL2 has not still been achieved.

In addition, rhIL2 shows poor stability in vitro and a short plasmatic half-life and fast renal clearance in vivo.

It is known also a composition comprising a chelating agent dietylenetriaminepentaacetic acid (DTPA), a protein and a metallic cation (WO96/15816). In this document said chelating agent is bound to said metallic cation and conjugated, site specifically, to the N-terminus of said protein that is rhIL2. The metallic cation is radioactive and is gallium-67, indium-111 or technetium-99m. However, the use of these gamma-emitting radionuclides suffers from many disadvantages, especially when coupled to biologically active proteins. For example, the resolution of the gamma-camera is less than that of a PET tomograph and it does not permit to quantify the uptake in target lesions. Moreover, all the studies carried out have shown that, apart from technetium, the other radioisotopes do not have optimal characteristics for use in humans, both in terms of resolution and radiation dose to the patient. Finally, the conjugation of DTPA to N-terminus of the IL2 may significantly reduce its binding to IL2R.

Chianelli M et al., in 1997 (Nucl Med Biol. 1997 August; 24(6):579-86) described technetium-99m-labelled IL2 using an N₃S chelating agent. However, ^{99m}Tc is an isotope for gamma-camera imaging and not for PET, and N₃S agents also bind to the N-terminus of the protein reducing its binding to specific receptor. Indeed, suboptimal biodistribution in humans was observed. Finally, this method implies to perform first the labelling of N₃S with^{99m}Tc and then its conjugation to IL2, thus making the preparation of the radiopharmaceutical very tedious, expensive and time consuming.

D'Alessandria et al., in 2009 (Mol Imaging Biol. 2010 October; 12(5):539-46) described the synthesis of ^{99m}Tc-Hynic-IL2 for in vivo imaging of activated T lymphocytes. They described the pre-conjugation of the protein with succimidyl-6-hydrazinopyridine-3-carboxilate (HYNIC-NHS) as a bifunctional chelating agent and tricine as coligand obtaining a ternary ligand system. Also in this case, as for N₃S agents, HYNIC binds the N-terminus of the molecule and it's likely to interfere with IL2 binding to its receptor. Furthermore, the use of tricine could lead to the formation of intermediate radioactive species or aggregates with high accumulation in the liver, thus reducing the bioavailability of the radiopharmaceutical in the circulation.

Recently, the availability of ⁶⁸Ge/⁶⁸Ga generators allowed researchers to develop many ⁶⁸Ga-radiolabelled radiopharmaceuticals for PET imaging (such as ⁶⁸Ga-DOTA-NOC/⁶⁸Ga-DOTA-TOC/⁶⁸Ga-DOTA-TATE/⁶⁸Ga-PSMA and many others) in facilities that could not have access to a cyclotron.

In order to bind a metal radioiosotope to a protein stably the use of a chelator is required. One of the most commonly used chelators for ⁶⁸Ga is 1,4,7,10-tetraazacyclododecane-1,4,7,10-tetraacetic acid (DOTA). However, DOTA requires that the reaction is carried out at 95° C. and such a high temperature would cause protein degradation and/or loss of ternary and quaternary structure, especially for a large and thermolabile protein such as IL2.

For the abovementioned reasons, currently protein-based radiopharmaceuticals labelled with ⁶⁸Ga are not commercially available. In particular, no molecules are available to detect selectively interleukin-2 receptor expressing cells in vivo by PET imaging.

In the light of the above, it is therefore apparent the need to provide new compounds for PET imaging of interleukin-2 receptor positive cells, which are able to overcome the disadvantages of the known methods and compounds.

According to the present invention, a new ⁶⁸Ga-radiolabelled radiopharmaceutical for PET imaging of IL2R positive cells is provided which can be prepared at room temperature without the need of a dedicated syntheses modules. The present invention provides also a kit comprising stable ingredients for the preparation of the radiopharmaceutical. This kit allows to easily have ready-to-use ⁶⁸Ga-labelled IL2 with high stability in both 0.9% NaCl solution and human plasma, high specific activity, optimal receptor binding capacity (Kd in nanomolar range), at reasonable costs. The kit is user-friendly, since it is sufficient to resuspend the content of the kit and add ⁶⁸Ga eluate from the ⁶⁸Ge/⁶⁸Ga generator. After 10-15 minutes the radiopharmaceutical is ready for the injection without further purification.

The radiopharmaceutical according to the present invention can be used in the field of nuclear medicine, in particular for PET imaging of IL2R positive cells. In particular, the radiopharmaceutical according to the present invention can be advantageously used for locating or imaging in vivo activated T-lymphocytes in a subject. Specifically, it can be used to view infiltrations of activated T-lymphocytes in all inflammatory diseases involving activated T lymphocytes. Besides, it can be used also in the assessment of the response to an immunotherapy, for example in patients affected by metastatic melanoma or any other solid cancer that can be treated with immunotherapeutic drugs that stimulate T cell infiltration.

Given the short plasmatic half-life and poor stability of native IL2, according to the present invention the labelling of a derivative of human IL2, namely desalanyl-1, serine-125 human interleukin-2 (dsIL2) with ⁶⁸Ga has been tested. According to the present invention dsIL2 can be labelled with ⁶⁸Ga, when ds IL2 is conjugated with an appropriate chelating agent for radiometals at room temperature.

As mentioned above, it is known that most of the ⁶⁸Ga radiolabeled drugs need an incubation time with temperatures higher than 95° C. for their production. The use of ⁶⁸Ga radiolabeling is therefore not appropriate with large and thermolabile proteins such as interleukin-2. According to the present invention, a chelator which allows radiolabeling proteins at room temperature thus avoiding to heat the reaction mixture has been used. In particular, according to the present invention tris(hydroxypyridinone)-maleimide (THP-mal) has been used as chelator, which is able to efficiently bind both dsIL2 and ⁶⁸Ga at room temperatures.

In addition, the use of THP-mal, which binds to SH groups on the dsIL2 molecule, permits to overcome the limitation of other chelators that binds to the free NH2 group at the N-terminal of the protein. As reported by Collind et al. (J Biol Chem. 1987 Apr. 25; 262(12):5723-31) the NH2 group at the N-terminal of IL2 participates in the binding to the IL2 receptor. Therefore, the presence of a chelator in that portion of the molecule might interfere with the proper binding to the IL2R.

According to the present invention, it has been found that by using a chelator that binds to SH groups, like THP-mal, the affinity of the radiolabeled compound for its receptor is not affected. Furthermore, THP-mal can be coupled with ⁶⁸Ga at room temperature, thus avoiding exposure of IL2 to high temperature.

Other compounds can allow radiolabeling of proteins at room temperature. For example 1,4,7-triaza-cyclononane, 1-glutaric acid-4,7-acetic acid (NODAGA) and THP-NCS. Nevertheless, they all bind to the N-terminal residue of the protein.

The inventors had the intuition to test different chelating agents that could bind to different residues of IL2 (not NH2) in order to preserve its receptor binding activity. After several attempts the inventors discovered that a chelating agent that binds SH groups at room temperature (THP-mal) is suitable for binding proteins with Cysteine residues. However, since native human IL2 has 3 Cysteine residues in position 58, 105 and 125, the THP-mal could bind to several of these cysteines. The inventors had the second intuition, i.e. the use of a mutated form of human IL2, namely dsIL2, that has only 2 Cysteine residues in position 58, 105 forming a disulphide bond. The cysteine in position 125 has been substituted with a Serine. Surprisingly, the inventors set up a perfect experimental condition leading to conjugation of only one molecule of THP-mal between Cys-58 and Cys-105, as demonstrated by HPLC, MALDI-TOF and SDS-PAGE, without interfering with IL2R binding site as shown by in vitro binding assay in the example.

According to the present invention, a kit and a procedure to radiolabel dsIL2 with ⁶⁸Ga at room temperature, using THP-mal as a chelator, are provided. The kit of the present invention is suitable to be used in every center of nuclear medicine equipped with a ⁶⁸Ga generator. Particularly, the present invention provides a cold lyophilized, sterile and pyrogens-free, mono-use kit for the GMP synthesis of radiolabelled desalanyl-1, serine-125 human interleukin-2 (dsIL2) with gallium-68 (⁶⁸Ga) at room temperature using tris(hydroxypyridinone)-maleimide (THP-mal) and its subsequent conversion to the ⁶⁸Ga-THP-dsIL2.

The methodology according to the present invention is expected to overcome major limitations of known IL2 radiolabelling methods or compounds. These limitations are represented by low labelling efficiency with other chelating agents, reduced IL2R binding, altered biodistribution, degradation of IL2 due to high temperature, time consuming labelling and need of post-labelling purification when using other radioisotopes or labelling methods.

In fact, the present invention provides a kit based on THP-mal-dsIL2 that can be radiolabelled at room temperature without the need of a dedicated syntheses modules. In addition, the high labelling efficiency obtained for ⁶⁸Ga-THP-mal-dsIL2 radiopharmaceutical according to the present invention allows avoiding a final purification. Therefore, the radiopharmaceutical according to the present invention can be prepared in laboratories which are not provided with a dedicated radiopharmaceuticals synthesis module. Finally, the kit according to the present invention is easy to use and it lowers radiopharmaceutical production times by more than 50%.

THP-mal-dsIL2 has been tested by using different THP-mal:dsIL2 ratios up to 40:1 and it has been found that all the ratio were suitable for conjugation and labeling. Higher ratio could also be used effectively. Then ⁶⁸Ga-THP-mal-dsIL2 has been prepared wherein dsII2 maintains its biological activity and receptor binding activity. A stability of the radiopharmaceutical according to the present invention up to 3 hours has been also observed. According to in vitro experiments, preparation of ⁶⁸Ga-THP-dsIL2 needs approximately no more than 20 minutes including quality controls. Indeed, after elution of ⁶⁸Ga from the generator, it is sufficient to add this solution to the reaction vial of the kit and incubate for 10-15 minutes at room temperature before performing due quality controls.

In addition, in vivo experiments are also described in the example, wherein, in normal Balb/C mice, the ⁶⁸Ga-THP-dsIL2 showed favorable biodistribution with higher kidneys-to-liver ratio, if compared with other radiolabeled IL2 radiopharmaceuticals.

Therefore, it is a specific object of the present invention a radiopharmaceutical compound for the imaging or for locating IL2R positive cells, such as activated T lymphocytes, said compound comprising or consisting of a protein able to bind IL2R, such as recombinant human desalanyl-1, serine-125 human IL2 or other forms of IL2, fragments or mutants thereof having at least one Cysteine residue, which are capable to bind IL2R alpha and/or beta, wherein said protein is labelled with a radioisotope (more in particular radiometals) chosen from the group consisting of positron-emitting radioisotopes, such as ⁶⁸Ga, ⁶⁴Cu, and other positron-emitting metals, by a chelator, the chelator being a chelator with a reactive group able to bind SH groups and to chelate the radioisotope, at room temperature.

For example other forms of IL2, which are capable to bind IL2R alpha and/or beta are mutant Interleukin-2v, F42K mutant form of IL2, F8-IL2, mutant human interleukin 2((88)Arg, (125)Ala).

In particular, the chelator can comprise a core of tris-(hydroxypyridinone) (THP), such as the preferred chelator tris(hydroxypyridinone)-maleimide,1,4,7-triaza-cyclononane (THP-mal) having formula C44H57N9O13 (and molecular weight of 919,41), the chelator maleimide-1-glutaric acid-4,7-acetic acid (NODAGA-mal) or any other chelator for radiometals that allows binding to SH groups of proteins and the radiometal at room temperature, for example desferroxiamine-maleimide (DFO-mal) or maleimide-diethylene triamine pentaacetic acid (DTPA-mal).

According to an embodiment of the present invention, the protein can be recombinant human desalanyl-1, serine-125 human interleukin-2 and said chelator can be THP-mal.

According to the present invention, the chelator is not bound to NH terminal group of the protein able to bind interleukin-2 receptors, the chelator being bound to a SH group of the protein. In particular, according to the present invention, a method has been set up for attaching only one chelator to one SH group of the protein, in other words the protein is conjugated with only one chelator molecule, this avoiding large modifications of the protein and preserve its receptor binding activity. The method of the invention also avoids any interference that might derive from the presence of a chelating agent at the N-terminal of the IL2 molecule, where the ligand interacts with its receptors.

The present invention concerns also a precursor compound of the radiopharmaceutical compound as defined above, said precursor comprising or consisting of a protein able to bind interleukin-2 receptors, such as recombinant human desalanyl-1, serine-125 human interleukin-2 or other forms of interleukin-2, fragments or mutants thereof having at least one Cysteine residue, which are capable to bind interleukin-2 receptors alpha and/or beta, said protein being conjugated with a chelator, the chelator being a chelator with a reactive group able to bind SH groups and to chelate a radioisotope (more in particular radiometals) chosen from the group consisting of positron-emitting radioisotopes, such as ⁶⁸Ga, ⁶⁴Cu, and other positron-emitting metals at room temperature.

As mentioned above, the chelator can comprise a core of tris-(hydroxypyridinone) (THP) such as the chelator tris(hydroxypyridinone)-maleimide,1,4,7-triaza-cyclononane (THP-mal) having formula C44H57N9O13 (and molecular weight of 919,41), maleimide-1-glutaric acid-4,7-acetic acid (NODAGA-mal) or any other chelator for radiometals that allows binding to SH groups of proteins and radiometals, such as ⁶⁸Ga, at room temperature.

According to an embodiment, the precursor compound can comprise recombinant human desalanyl-1, serine-125 human interleukin-2 as protein and THP-Mal as chelator.

In addition, the present invention concerns a radiopharmaceutical composition comprising or consisting of the radiopharmaceutical compound as defined above, in association with one or more excipients and/or adjuvants.

A further object of the present invention is a pharmaceutical composition comprising or consisting of the precursor compound as defined above, in association with one or more excipients and/or adjuvants.

In particular the excipients and/or adjuvants of said radiopharmaceutical composition or pharmaceutical composition can comprise a biocompatible carrier medium, such as a buffer, for example phosphate-buffered saline (PBS) and sodium-dodecyl-sulphate (SDS); and/or one or more additives chosen from the group consisting of antimicrobial preservative, pH-adjusting agent or filler.

By the term “antimicrobial preservative” is meant an agent, which inhibits the growth of potentially harmful micro-organisms such as bacteria, yeasts or molds. The antimicrobial preservative may also exhibit some bactericidal properties, depending on the dose. The main role of the antimicrobial preservative(s) of the present invention is to inhibit the growth of any such micro-organism in the radiopharmaceutical composition. The antimicrobial preservative can be chosen from the group consisting of parabens, such as methyl, ethyl, propyl or butyl paraben or mixtures thereof; benzyl alcohol; phenol; cresol; cetrimide and thiomersal; preferably parabens.

The pH-adjusting agent can be chosen from the group consisting of pharmaceutically acceptable buffers, such as tricine, phosphate buffer or TRIS (i.e. tris(hydroxymethyl)aminomethane) or mixtures thereof; pharmaceutically acceptable bases such as sodium carbonate, sodium bicarbonate or mixtures thereof; preferably a phosphate buffer, more preferably a phosphate buffer with 10% SDS. For ⁶⁸Ga-THP-dsIL2, a preferred buffer is phosphate buffer with 10% SDS.

By the term “filler” is meant a pharmaceutically acceptable bulking agent, which may facilitate material handling during product production. Suitable fillers include inorganic salts, such as sodium chloride; water-soluble sugars; sugar alcohols, such as sucrose, maltose, mannitol or trehalose.

The radiopharmaceutical composition according to the present invention can be in the form of an injectable composition.

The present invention concerns also a radiopharmaceutical compound as defined above, a precursor compound as defined above, a radiopharmaceutical or pharmaceutical composition as defined above for use in medical field.

A further object of the present invention is a radiopharmaceutical compound as defined above or a radiopharmaceutical composition as defined above for use in the evaluation of T cell infiltration in active inflammatory and infective diseases, such as autoimmune diseases, vasculitis, sarcoidosis, tuberculosis and others, and in solid tumors characterized by a T-cell infiltration, such as metastatic melanoma.

The present invention concerns also a radiopharmaceutical compound as defined above or a radiopharmaceutical composition as defined above for use in in vivo diagnostic methods for locating or imaging interleukin-2 receptor positive cells in a subject, such as by PET imaging diagnostic method.

A further object of the present invention is a use of the radiopharmaceutical compound as defined above or radiopharmaceutical composition as defined above as imaging agent in PET imaging.

The present invention concerns also a method for obtaining a radiopharmaceutical compound as defined above or a radiopharmaceutical composition as defined above, said method comprising:

a) conjugating a protein able to bind IL2R, such as recombinant human desalanyl-1, serine-125 human interleukin-2 or other forms of IL2, fragments or mutants thereof having at least one Cysteine residue, which are capable to bind interleukin-2 receptors alpha and/or beta, with a chelator able to bind SH groups not interfering with IL2 binding to IL2R and to chelate a radioisotope (or in particular a radiometal) chosen from the group consisting of positron-emitting radioisotopes, such as ⁶⁸Ga, ⁶⁴Cu, or others, at room temperature, in order to obtain a precursor compound of the radiopharmaceutical compound.

The method according to the present invention can further comprise:

b) radiolabelling with a radioisotope (in particular radiometals) chosen from the group consisting of positron-emitting radioisotopes, such as ⁶⁸Ga, or ⁶⁴Cu, or others, the precursor compound obtained in step a), at room temperature.

According to the method of the present invention, the chelator can comprise a core of tris-(hydroxypyridinone) (THP) such as the chelator tris(hydroxypyridinone)-maleimide,1,4,7-triaza-cyclononane (THP-Mal) having formula C44H57N9O13 (and molecular weight of 919,41), maleimide-1-glutaric acid-4,7-acetic acid (NODAGA-mal) or any other chelator for radiometals that allows radiometal radiolabeling at room temperature without interfering with IL2 binding to IL2R.

According to an embodiment, the protein able to bind to interleukin-2 receptors can be recombinant human desalanyl-1, serine-125 human interleukin-2 and said chelator can be THP-mal.

The present invention concerns also a kit for the preparation of a radiopharmaceutical compound as defined above or a radiopharmaceutical composition as defined above, said kit can comprise or consist of:
a first vial comprising a precursor compound as defined above or pharmaceutical composition as defined above.

The kit according to the present invention can further comprise:
a second vial comprising a buffer having pH ranging from 5 to 5.5, such as an ammonium acetate water solution or HEPES.

In addition, the kit can further comprise luer-lock compatible adapter or any other adapter apt to allow the placement of a vial containing said precursor on a pre-made cassette of any automated synthesizer of radiopharmaceuticals that utilizes disposable cassettes.

On the basis of the above the present invention concerns a kit to synthesize a tool for locating or imaging a region containing an in vivo accumulation of activated T-lymphocytes in a subject. In particular, the tool can be administrated to a subject in an effective amount in order to locate or image activated T-lymphocytes. In fact said tool comprises a targeting polypeptide, which is capable of binding specifically to interleukin-2 receptors of activated T-lymphocytes and carries one or more physiologically compatible imaging agents. Therefore, the tool is able to locate or image said accumulation of activated T-lymphocytes.

According to an embodiment, the present invention concerns a method in which lyophilized THP-mal-dsIL2 is dispensed into sterile, pyrogens free vials, under vacuum or nitrogen atmosphere, as a simple kit to be reconstituted with freshly eluted ⁶⁸Ga from a ⁶⁸Ge/⁶⁸Ga generator in order to produce ⁶⁸Ga-THP-mal-dsIL2 which is sterile, pyrogens-free, stable and capable to bind to IL2 receptors.

Activated T-lymphocytes can be present in any tissue of body including and can be caused by different disorders such as cancers, abscesses, inflammation, autoimmunity, tissue transplant rejection.

As mentioned above, the radiopharmaceutical composition according to the present invention can be administered to the human body together with a biocompatible carrier medium, for example a buffer such as PBS+SDS, without toxicity or undue discomfort.

The radiopharmaceutical compositions of the present invention can be suitably supplied in a clinical grade syringe or in a container, which is provided with a seal and is suitable for single or multiple puncturing with a hypodermic needle (e.g. a crimped-on septum seal closure) whilst maintaining sterile integrity. Such containers may contain single doses (a “unit dose”) or multiple patient doses. Suitable containers comprise a sealed vessel, which permits maintenance of sterile integrity and/or radioactive safety, whilst permitting addition and withdrawal of solutions by syringe. A preferred container is a septum-sealed vial, wherein the gas-tight closure is crimped on with an overseal (typically of aluminum). Such containers have the additional advantage that the closure can withstand vacuum if desired for example to change the headspace gas or degas solutions.

Radiopharmaceutical syringes are designed to contain a single human dose, or “unit dose” and are therefore preferably a disposable syringe or other syringe suitable for clinical use. Such syringes may optionally be provided with a syringe shield to protect the operator from radioactive dose. Suitable such radiopharmaceutical syringe shields are known in the art, and various designs are commercially available, and preferably comprise either lead or tungsten.

The radiopharmaceuticals of the present invention may be prepared under aseptic manufacture conditions to give the desired sterile, pyrogen-free product. The radiopharmaceuticals may also be prepared under non-sterile conditions, followed by terminal sterilization using e.g. gamma-irradiation or membrane filtration (sometimes called sterile filtration).

The present invention is described by an illustrative, but not limitative way, according to preferred embodiments thereof, with particular reference to the enclosed drawings, wherein:

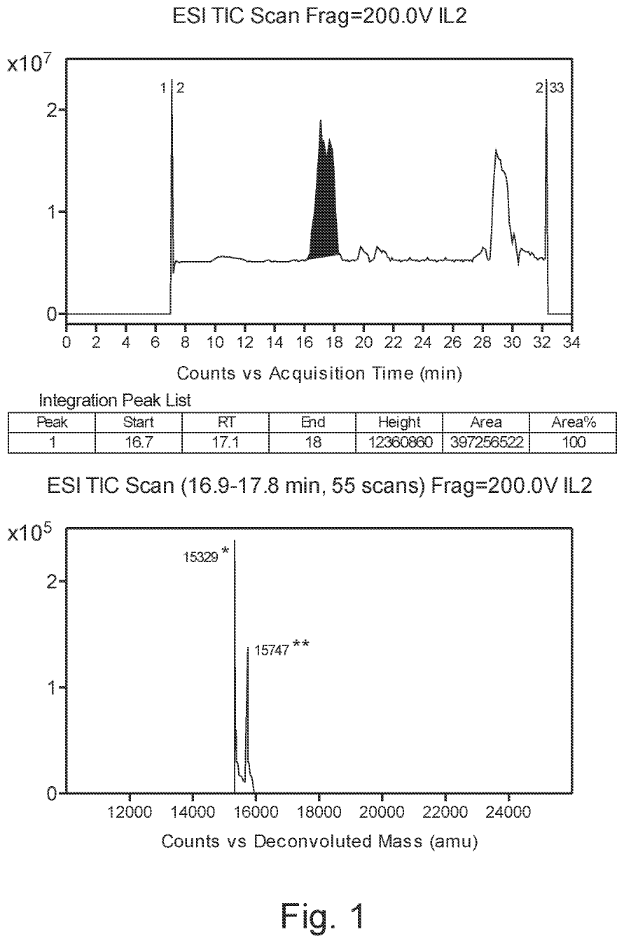

FIG. 1 shows ESI characterization of unconjugated dsIL2 (*) and THP-mal conjugated dsIL2 (**). This graph proves that the difference in the mass is caused by the addition of a THP-mal group after the conjugation step.

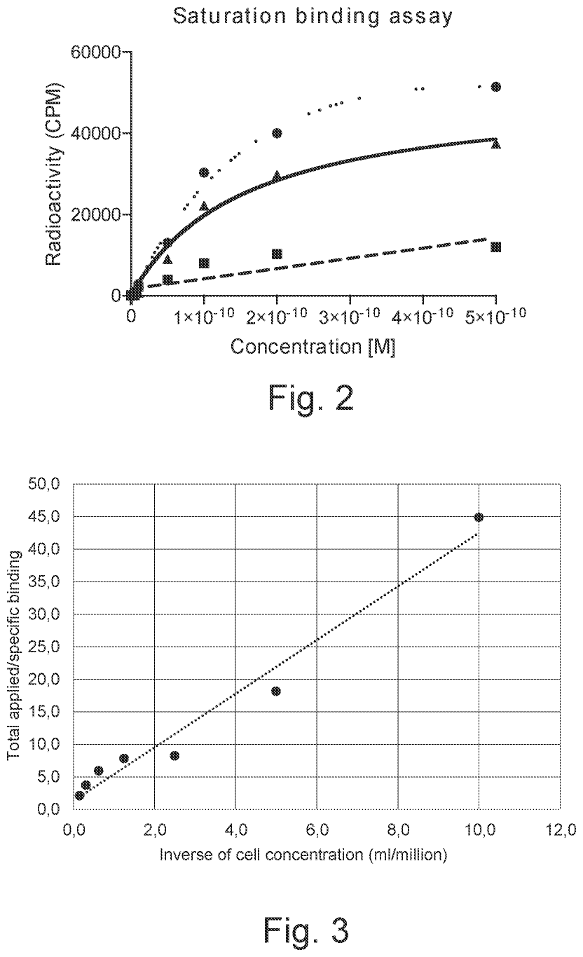

FIG. 2 shows the results of the saturation binding assay of radiolabeled IL2 on activated T cells; the graph shows total binding (circles), unspecific binding (squares) and specific binding (triangles) to IL2 receptor.

FIG. 3 shows the results of the Immunoreactive fraction assay of radiolabeled IL2 on activated T-cells; The graph is an inverted plot from which the formula to calculate the % IRF has been extrapolated.

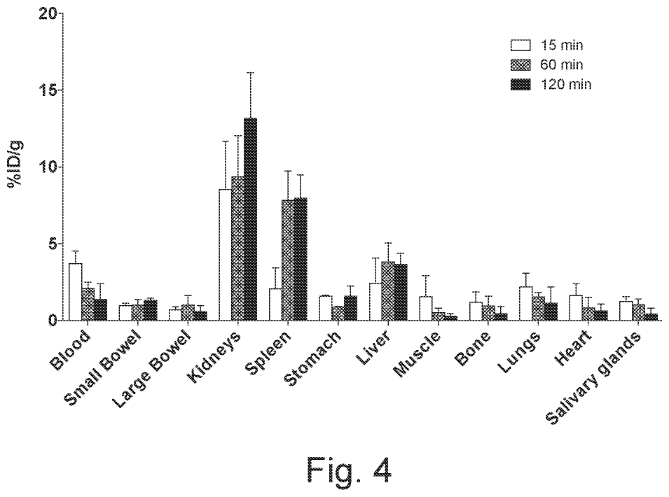

FIG. 4 shows the results of the biodistribution of radiolabeled IL2 in normal BALB/C mice; The graph shows the percentage of injected dose per gram (% ID/g) in major organs after 15 (white), 60 (grey) and 120 (black) minutes from injection.

### EXAMPLE 1: METHOD OF RADIOLABELLING DSIL2 WITH ⁶⁸GA AND PREPARATION OF A LYOPHILIZED KIT

Experiments Performed to Set-Up a Kit for Radiolabelling Interleukin-2 with ⁶⁸Ga

Several experiments have been set up to find the best concentrations of dsIL2, THP-mal and buffers to obtain the best conjugation and labelling conditions.

THP-mal:dsIL2 ratios of 5:1, 10:1, 20:1, 40:1 (i.e. from 5:1 to 40:1) have been tested and it was found that for all of them the chelating agent binds to the dsIL2 molecule, generating a single species of precursor, as revealed by liquid chromatography-Mass Spectroscopy analysis (ESI) shown in FIG. 1. This is a very relevant finding, since, this allow us to prevent the formation of different conjugates containing more than one THP-mal group, thus potentially reducing the affinity of the radiopharmaceutical for its receptor. Therefore, it is possible to produce only one species, and not a mixture of THP-mal-dsIL2 conjugates with different structures. The kit, which contains THP-mal-dsIL2, can be reconstituted with 0.5-1 ml of generator eluate containing up to 300 MBq of ⁶⁸Ga in HCl 0.1 M, at room temperature, gently mixing for 10 minutes, with or without using a syntheses module. The content of the kit is sterile and sterility will be preserved in each passage and can be directly injected into the patient. The amount of ⁶⁸Ga-THP-mal-dsIL2 obtained with 300 MBq of ⁶⁸Ga can be used for 1 or 2 patients within 1 h from the syntheses.

Preparation of a Kit for Radiolabelling Interleukin-2 with ⁶⁸Ga

A lyophilized kit composed of 2 vials and an adapter has been prepared using:

a vial 1 containing 160 μg of lyophilized THP-mal-dsIL2;

a vial 2 containing 300 μl of an ammonium acetate solution in H2O (pH=5); and optionally

a luer-lock compatible adapter with which vial 1 can be directly placed on a pre-made cassette of any automated synthesizer of radiopharmaceuticals.

For the preparation of vial 1, THP-mal (a THP derivative, tris(hydroxypyridinone)-maleimide, Chemical Formula C44H57N9O13) in a 20:1 ratio is added dropwise, over 10 minutes with gentle mixing, to a solution containing 160 μg of dsIL2 in mannitol and sodium dodecyl sulphate, buffered with monobasic and dibasic sodium phosphate to a pH of 7.5 (range 7.2 to 7.8). After 1 h incubation at room temperature, the product is purified by size exclusion chromatography using 0.9% NaCl as eluent and the conjugate is filtered through a 0.22 μm filter and then lyophilized under nitrogen atmosphere in a 10 ml crimped, glass vial compatible with any automated synthesizer of radiopharmaceuticals that does not use pre-made cassettes.

For the preparation of vial 2, 300 μl of an ammonium acetate solution 0.1 M (pH=5) are filtered through a 0.22 μm filter and placed in a 1 ml crimped glass vial under nitrogen atmosphere.

For ⁶⁸Ga labelling, the content of vial 2 is transferred to vial 1. After gently mixing, 0.5-1 ml of ⁶⁸Ga eluate in HCl 0.1 N (100-300 MBq, in this case approximately 300 MBq, of freshly eluted ⁶⁸Ga in HCl 0.1 M) are added to vial 1 containing 160 μg of THP-dsIL2, and ammonium acetate (0.1 M, pH=5). After 10 minutes incubation at room temperature, the solution in vial 1 is ready to be injected into the patient. To use the kit with an automated synthesizer, it is possible to transfer the content of vial 2 into vial 1 and then, through the luer-lock adapter, it is possible to mount the vial in a virgin cassette to directly elute ⁶⁸Ga from the generator into vial 1. Alternatively, this can be performed by eluting the generator with a peristaltic pump directly into the vial containing the precursor.

This procedure will produce ⁶⁸Ga-THP-mal-dsIL2 with retention of its biological activity and receptor binding activity.

At the end of the incubation, the solution is filtered with a 0.22 μm filter (Millex GV, Millipore) and quality controls can be performed.

Quality Controls

Quality controls are performed by both reverse phase HPLC (RP-HPLC) and size exclusion HPLC.

RP-HPLC can be performed using a kinetex C18 column (Phenomenex) and a gradient of H₂O and ACN as mobile phases (0-5 min 5% ACN; 5-15 50% ACN; 15-25 95% ACN; 25-35 5% ACN). Size exclusion HPLC can be performed using a Yarra column (Phenomenex) and 0.1 M phosphate buffer as mobile phase (isocratic).

Stability of the radiopharmaceutical has been tested against 0.9% NaCl solution and human serum donated by normal volunteers after signing written consent up to 3 h.

Radiochemical purity is determined using ITLC-SG strips developed with 0.1M HCl to quantify levels of ionic ⁶⁸Ga, and ITLC-SG strips developed with 1:1 MeOH:1 M NH4OAc to quantify colloidal ⁶⁸Ga-hydroxide plus ionic ⁶⁸Ga. The strips can be analyzed with a Bioscan AR-2000 radiochromatogram scanner fitted with high-resolution collimator. Negligible amount of free or colloidal ⁶⁸Ga should be observed (<5%).

Saturation Binding Assay

In order to verify the receptor binding capacity of radiolabelled IL2, T-cells were isolated from peripheral blood mononuclear cells (PBMNCs) from healthy donors (after signing written consent) by centrifugation on a standard Ficoll/Hyplaque density gradient. Cells were cultured for 48 to 72 h at 106/mL in complete culture medium and 1 kg/mL purified phytohemagglutinin (PHA) (Mirux) at 37° C. Before the assays, cells were incubated for 60 min at 37° C. in RPMI medium to remove endogenous IL2 from the cell-surface IL2R and then washed twice and resuspended in iced 1% BSA phosphate-buffered saline (PBS) containing 0.01% sodium azide (4° C.).

Then, 3×10⁶ cells were placed in triplicate in Eppendorf vials and incubated with decreasing concentrations of ⁶⁸Ga-THP-mal-dsIL2 for 1 hour at 4° C., to calculate total binding curve. The same experiment was performed in the presence of a 100 fold molar excess of unlabelled dsIL2 to each vial, to calculate non-specific binding. At the end of the incubation time, the cells were washed twice with 0.5 ml of PBS. After centrifugation, cell pellets and the supernatants were counted separately in a single-well gamma counter (Perkin Elmer). Data were analyzed using Prism Graphpad software, as shown in FIG. 2, and revealed a Kd value of 1.79 nM.

Immunoreactive Fraction (IRF) Assay

IRF assay was performed as described by Lindmo et al. Briefly, cells were seeded in Eppendorf vials (from 8×106/mL to 0.4×106/mL) and in each vial ⁶⁸Ga-THP-mal-dsIL2 was added at constant concentration (10 nM). After 1 h incubation at 4° C., the vials were centrifuged at 13000 rpm (5000 g) for 3 minutes and the supernatant was collected. This step was repeated after washing the pellet with 0.5 ml of PBS.

Radioactivity associated with pellets and supernatants was then determined by counting each vial with a single-well gamma counter (Perkin Elmer). Data were analyzed using Prism Graphpad software and an IRF of 78.4% was obtained, as shown on FIG. 3, demonstrating that the majority of dsIL2 is radiolabelled and capable to bind to its receptor.

Biodistribution in Normal BALB/C Mice

Biodistribution studies were performed in 12 normal BALB/C mice. Each animal received an intravenous injection (tail vein) of 0.55 MBq (in 1000 μl) of ⁶⁸Ga-THP-mal-dsIL2 according to the present invention. After 15, 60 and 120 minutes from the injection, four animals per time point were sacrificed to collect major organs and blood (collected samples included intestine, kidneys, spleen, stomach, liver, muscle, bone, lungs, heart and salivary glands). Each sample was weighted and counted with a single-well gamma counter to determine radioactivity. Data were expressed as percentage of injected dose per gram of tissue (% ID/g). FIG. 4 shows the results of the biodistribution that highlight a higher % ID/g in kidneys, where native human IL2 is normally metabolized and lower % ID/g in the liver, if compared with other radiolabeled IL2 species. This strengthen the hypothesis that binding of the THP-mal chelating agent to SH groups instead of the NH2 at the N-terminus, confers to the radiolabeled IL2 a more favorable biodistribution.

1. Radiopharmaceutical compound for the imaging or for locating interleukin-2 receptor positive cells, such as activated T lymphocytes, said compound comprising or consisting of a protein labelled with a radioisotope by a chelator, wherein
said protein is a protein consisting of recombinant human desalanyl-1, serine-125 human interleukin-2, a fragment or a mutant thereof having at least one Cysteine residue, which is capable to bind interleukin-2 receptors alpha and/or beta;
said radioisotope is a positron-emitting radioisotope, such as ⁶⁸Ga and ⁶⁴Cu; and
said chelator is a chelator with a reactive group able to bind a SH group of said recombinant human desalanyl-1, serine-125 human interleukin-2, fragment or mutant thereof and to chelate the radioisotope, at room temperature.

2. Radiopharmaceutical compound according to claim 1, wherein said chelator is a maleimide-coupled metal chelator such as tris(hydroxypyridinone)-maleimide,1,4,7-triaza-cyclononane (THP-mal) having formula C44H57N9O13 or maleimide-1-glutaric acid-4,7-acetic acid (NODAGA-mal).

3. Radiopharmaceutical compound according to claim 1, wherein said protein is recombinant human desalanyl-1, serine-125 human interleukin-2 and said chelator is THP-mal.

4. Precursor compound of the radiopharmaceutical compound as defined by claim 1, said precursor comprising or consisting of a protein conjugated with a chelator, wherein
said protein is a protein consisting of recombinant human desalanyl-1, serine-125 human interleukin-2, a fragment or a mutant thereof having at least one Cysteine residue, which is capable to bind interleukin-2 receptors alpha and/or beta; and
said chelator is a chelator with a reactive group able to bind a SH group of said recombinant human desalanyl-1, serine-125 human interleukin-2, fragment or mutant thereof and to chelate a positron-emitting radioisotope, such as ⁶⁸Ga and ⁶⁴Cu, at room temperature.

5. Precursor compound according to claim 4, wherein said chelator is a maleimide-coupled metal chelator such as tris(hydroxypyridinone)-maleimide,1,4,7-triaza-cyclononane (THP-mal) having formula C44H57N9O13 or maleimide-1-glutaric acid-4,7-acetic acid (NODAGA-mal).

6. Precursor compound according to claim 4, wherein said protein is recombinant human desalanyl-1, serine-125 human interleukin-2 and said chelator is THP-mal.

7. Radiopharmaceutical composition comprising or consisting of the radiopharmaceutical compound as defined in claim 1, in association with one or more excipients and/or adjuvants.

8. Pharmaceutical composition comprising or consisting of the precursor compound as defined in claim 4, in association with one or more excipients and/or adjuvants.

9.-14. (canceled)

15. A method of in vivo evaluating T cell infiltration in active inflammatory and infective disease in a subject, the method comprising administering to the subject a radiopharmaceutical compound according to claim 1.

16. A method of in vivo locating or imaging interleukin-2 receptor positive cells in a subject, the method comprising administering to the subject a radiopharmaceutical compound according to claim 1.

17. An imaging agent for PET imaging comprising the radiopharmaceutical compound according to claim 1.

18. Method for obtaining a radiopharmaceutical compound according to claim 1, said method comprising:
a) conjugating a protein with a chelator in order to obtain a precursor compound of the radiopharmaceutical compound, wherein
said protein is a protein consisting of recombinant human desalanyl-1, serine-125 human interleukin-2, a fragment or a mutant thereof having at least one Cysteine residue, which is capable to bind interleukin-2 receptors alpha and/or beta; and
said chelator is a chelator with a reactive group able to bind a SH group of said recombinant human desalanyl-1, serine-125 human interleukin-2, fragment or mutant thereof and to chelate a positron-emitting radioisotope, such as ⁶⁸Ga and ⁶⁴Cu, at room temperature.

19. Method according to claim 18, further comprising:
b) radiolabelling with a positron-emitting radioisotope, such as ⁶⁸Ga and ⁶⁴Cu, the precursor compound obtained in step a), at room temperature.

20.-21. (canceled)

22. Kit for the preparation of a radiopharmaceutical compound according to claim 1, said kit comprising or consisting of:
a first vial comprising a precursor compound comprising or consisting of a protein conjugated with a chelator, wherein said protein is a protein consisting of recombinant human desalanyl-1, serine-125 human interleukin-2, a fragment or a mutant thereof having at least one Cysteine residue, which is capable to bind interleukin-2 receptors alpha and/or beta; and said chelator is a chelator with a reactive group able to bind a SH group of said recombinant human desalanyl-1, serine-125 human interleukin-2, fragment or mutant thereof and to chelate a positron-emitting radioisotope, such as ⁶⁸Ga and ⁶⁴Cu, at room temperature, or a pharmaceutical composition comprising or consisting of the precursor compound in association with one or more excipients and/or adjuvants.

23.-24. (canceled)

25. Method according to claim 15, comprising PET imaging.

26. Method according to claim 16, comprising PET imaging.

27. A method of in vivo evaluating T cell infiltration in active inflammatory and infective disease in a subject, the method comprising administering to the subject a radiopharmaceutical composition according to claim 7.

28. A method of in vivo locating or imaging interleukin-2 receptor positive cells in a subject, the method comprising administering to the subject a radiopharmaceutical composition according to claim 7.

29. Method according to claim 27, comprising PET imaging.

30. Method according to claim 28, comprising PET imaging.
